(19) United States
(12) Patent Application Publication
(10) Pub. No.: US 2025/0098608 A1
(43) Pub. Date: Mar. 27, 2025

(54) MUTANTS OF BACILLUS AND METHODS FOR THEIR USE

(71) Applicant: Bayer CropScience LP, St. Louis, MO (US)

(72) Inventors: Damian CURTIS, Davis, CA (US); Magalie GUILHABERT, Davis, CA (US); David J. LARSEN, Sacramento, CA (US); Donglan TIAN, Davis, CA (US)

(21) Appl. No.: 18/649,596

(22) Filed: Apr. 29, 2024

Related U.S. Application Data
(63) Continuation of application No. 16/344,991, filed on Apr. 25, 2019, now abandoned.
(60) Provisional application No. 62/414,339, filed on Oct. 28, 2016.

(51) Int. Cl. A01H 3/00; A01N 63/22; C12N 1/20; C12R 1/07

(52) U.S. Cl. CPC A01H 3/00; A01N 63/22; C12N 1/205; C12R 2001/07

(57) ABSTRACT

The present invention relates to a composition comprising a biologically pure culture of plant growth promoting mutants of Bacillus firmus strain I-1582. The present invention also provides a method of treating a seed to promote plant growth, wherein the method comprises applying such mutants to the plant, to a part of the plant and/or to a locus of the plant.

### CROSS-REFERENCE TO RELATED APPLICATIONS

This application claims the benefit under 35 U.S.C. Section 119 (c) of U.S. Provisional Patent Application No. 62/414,339, filed Oct. 28, 2016. The contents of the aforementioned patent application is hereby incorporated by reference in its entirety.

### TECHNICAL FIELD

The present invention relates to the field of bacterial mutants and their ability to enhance plant health, including yield.

### BACKGROUND

In crop protection, there is a continuous need for applications that improve the health of plants. Healthier plants generally result in higher yields and/or better quality of a plant or its products. In addition, due to their increased vigor, healthier plants show a better resistance to biotic and/or abiotic stress.

In order to promote plant health, fertilizers are employed worldwide, based on both inorganic and organic substances. A fertilizer may be a single substance or a composition, and is used to provide nutrients to plants. A major breakthrough in the application of fertilizers was the development of nitrogen-based fertilizer by Justus von Liebig around 1840. Fertilizers, however, can lead to soil acidification and destabilization of nutrient balance in soil, including depletion of minerals and enrichment of salt and heavy metals. In addition, excessive fertilizer use can lead to alteration of soil fauna as well as contaminate surface water and ground water. Further, unhealthful substances such as nitrate may become enriched in plants and fruits.

A possible alternative to fertilizer for advancing plant growth is plant-associated bacteria such as rhizobacteria. Such bacteria are associated with many, if not all, plant species. The mechanism behind the effect of plant-associated bacteria on plant growth is still open to speculation. Ryu, et al., (Proc. Natl. Acad. Sci. U.S.A. 100, 4927-4932) have suggested that among rhizobacteria, which colonize roots, some strains regulate plant growth via releasing 2, 3-butanediol and/or acetoin.

There remains a need to provide alternative means of advancing the growth of a plant and improving its health. This includes a need for improved plant growth-promoting rhizobacteria that also control pests, such as nematodes.

### SUMMARY

To meet this need, Applicant developed plant growth-promoting mutants of Bacillus firmus strain I-1582, a strain known for its excellent nematode control activity. The present invention is directed to mutants of Bacillus firmus strain I-1582 that retain the nematode control properties of the parent strain and have enhanced plant growth-promotion capabilities. The invention also encompasses methods of generating such mutants.

The present invention is directed to a composition comprising plant-growth promoting mutants of a biologically pure culture of a Bacillus firmus strain I-1582, such as Bacillus firmus strain NRRL B-67003 or Bacillus firmus strain NRRL B-67518. The present invention is also directed to a composition comprising a biologically pure culture of a Bacillus firmus strain NRRL B-67003, a Bacillus firmus strain NRRL B-67518, or a plant growth-promoting mutant strain derived from one or more of these strains. In some aspects, the composition comprises a fermentation product of a plant-growth promoting mutant of B. firmus I-1582, such as B. firmus strain NRRL B-67003, B. firmus strain NRRL B-67518, or a plant growth-promoting mutant strain derived therefrom.

In certain aspects, the fermentation product further comprises a formulation ingredient. The formulation ingredient may be a wetting agent, extender, solvent, spontaneity promoter, emulsifier, dispersant, frost protectant, thickener, and/or an adjuvant. In one embodiment, the formulation ingredient is a wetting agent. In other aspects, the fermentation product is a freeze-dried powder or a spray-dried powder.

The preexamplessent invention also provides a method of treating a plant to improve plant health, including increasing plant yield, and/or to control a plant pest, such as a nematode, by applying to the plant, to a part of the plant and/or to a locus of the plant, Bacillus firmus strain NRRL B-67003, Bacillus firmus strain NRRL B-67518, or a mutant strain derived from one or both of such strains. In some embodiments, the Bacillus firmus strain NRRL B-67003, the Bacillus firmus strain NRRL B-67518 or a mutant strain derived from one or more of such strains is applied in a composition comprising the Bacillus firmus strain NRRL B-67003, the Bacillus firmus strain NRRL B-67518, or a plant growth-promoting mutant strain derived from one or both of such strains. The composition may be a fermentation product of the Bacillus firmus strain NRRL B-67003, the Bacillus firmus NRRL B-67518, or a mutant strain derived therefrom.

In other embodiments, the method comprises applying the composition to seed of a plant.

In other embodiments, the method further comprises applying a chemical fungicide and/or chemical insecticide to a seed of a plant, to the plant or to the locus around the plant. In a particular embodiment, the chemical insecticide is clothianidin, cypermethrin, cthiprole, fipronil, fluopyram, flupyradifurone, imidacloprid, methiocarb, or thiodicarb. In another particular embodiment, the fungicide is metalaxyl, bitertanol, bixafen, bromuconazole, carbendazim, carpropamid, dichlofluanid, fenamidone, fenhexamid, fentin acetate, fentin hydroxide, fluopicolide, fluopyram, fluoxastrobin, fluquinconazole, fosctyl, iprodione, iprovalicarb, isotianil, metominostrobin, ofurace, pencycuron, penflufen, prochloraz, propamocarb, propineb, prothioconazole, pyrimethanil, spiroxamine, tebuconazole, tolylfluanid, triadimefon, triadimenol, triazoxide, trifloxystrobin, N-[5-chloro-2-(trifluoromethyl)benzyl]-N-cyclopropyl-3-(difluoromethyl)-5-fluoro-1-methyl-1H-pyrazole-4-carboxamide, or 2,6-dimethyl-1H,5H-[1,4]dithiino [2,3-c:5,6-c′]dipyrrole-1,3,5,7 (2H,6H)-tetrone. In one aspect of this embodiment, the compositions of the present invention are applied to a seed with clothianidin and/or metalaxyl.

The present invention further provides use of a composition comprising a biologically pure culture of a plant growth-promoting Bacillus firmus strain NRRL B-67003, Bacillus firmus strain NRRL B-67518 or a mutant strain derived therefrom for improving plant health, including plant yield, in useful plants. In one embodiment, the invention is directed to use of a composition comprising a fermentation product of Bacillus firmus strain NRRL B-67003, Bacillus firmus strain NRRL B-67518, or a mutant strain derived therefrom for improving plant health in useful plants. In yet another aspect of this embodiment, the composition also comprises an agriculturally acceptable carrier, such as a formulation ingredient.

In other aspects, the useful plants are selected from the group consisting of soybean, corn, sorghum, cotton and sugarbeet.

In yet another aspect, the composition comprises a seed of a useful plant coated with Bacillus firmus strain NRRL B-67003, Bacillus firmus strain NRRL B-67518, or a mutant strain derived from one or more of such strains. In one embodiment, the strain is applied at about 1×10⁵ to about 1×10⁸ colony forming units (CFU) per seed of any of the strains.

The present invention also provides a method for generating a mutant of one or more parent Bacillus firmus strains with improved plant growth promotion properties comprising the steps of (i) mutagenizing the one or more parent Bacillus firmus strains to create mutants, (ii) measuring production of indole acetic acid in the mutants, (iii) selecting one or more mutants with enhanced production of indole acetic acid compared to the one or more parent Bacillus firmus strains, and (iv) producing one or more fermentation products of a selected mutant. In one embodiment the parent Bacillus firmus strain is Bacillus firmus strain I-1582.

In another embodiment, the mutagenizing may be accomplished by chemical mutagenesis, by irradiation and/or by genome shuffling. In certain aspects of these embodiments step (i) is repeated one or more times, first on the parent and then on subsequent mutants, prior to the measuring and selection of mutants in steps (ii) and (iii).

In yet another embodiment, step (iv) is followed by further screening of selected mutants by measuring plant growth promotion abilities of such mutants and selecting mutants with improved plant growth promotion abilities compared to the parent strain or other predecessor mutants. Mutants having improved plant growth promotion abilities compared to the parent strain may be further mutagenized and selected for enhanced production of indole acetic acid and/or further improved plant growth promotion capabilities.

In another embodiment, step (ii) further comprises measuring enzymatic activity of protease and/or superoxide dismutase in the mutants and step (iii) further comprises selecting one or more mutants with enhanced production of indole acetic acid and enhanced enzymatic activity of protease and/or superoxide dismutase compared to the parent or any other predecessor Bacillus firmus strain. In another aspect of this embodiment, the mutagenesis step is repeated one or more times, with additional mutants being created from the first generation mutants and/or, if genome shuffling is used, from a combination of mutants from any generation or from a combination of mutants from any generation with the parent strain, and subsequent mutants are selected based on enhanced indole acetic acid production and/or enhanced enzymatic activity of protease and/or superoxide dismutase compared to the parent Bacillus firmus strain and/or to the mutants generated in any mutagenesis step.

In one embodiment, IAA production, protease activity and/or superoxide dismutase activity is increased by at least one fold, by at least two fold, by at least three fold, by at least four fold, by at least five fold, by at least six fold, by at least seven fold, by at least eight fold, by at least nine fold, or by at least ten fold over IAA production, protease activity and/or superoxide dismutase activity of one or more predecessor strains. A predecessor strain or strains may be the parent strain or strains from which a strain is directly derived or grandparent strains from which it is derived through multiple generations if multiple rounds of mutagenesis are undertaken. In a particular embodiment, activity is improved over all predecessor strains.

The present invention also provides a method for generating mutants having improved plant growth promotion properties from one or more parent strains of Bacillus species. Such strains may be from any Bacillus species, including B. subtilis, B. amyloliquefaciens, B. pumilus, and B. firmus. Such method involves (i) mutagenizing the one or more parent Bacillus strains to create mutants, (ii) measuring production in the mutants of a bacterial compound related to plant growth promotion, induced systemic resistance, and/or stress modulation, (iii) selecting one or more mutants with enhanced production of such bacterial compound compared to the one or more parent Bacillus strains, and (iv) producing one or more fermentation products of a selected mutant.

In one embodiment, the one or more parent strains are pre-screened for production of bacterial compounds related to plant growth promotion, induced systemic resistance and/or stress modulation and the measuring step is directed toward the bacterial compounds produced by the parent strain.

In one embodiment, the bacterial compound is a plant growth regulator, such as IAA, gibberellin, or cytokinin; a bacterial enzyme, such as 1-aminocyclopropane-1-carboxylate (ACC) deaminase or superoxide dismutase; and/or a compound that contributes to induced systemic resistance in plants, such as siderophores, salicylic acid and lipopolysaccharides. In one aspect of this embodiment measuring and selecting for enhanced production of a bacterial enzyme may be replaced by measuring and selecting for enhanced enzymatic activity of such bacterial enzyme.

In another embodiment, the mutagenizing is accomplished by chemical means, irradiation and/or genome shuffling. In yet another embodiment, the mutagenizing is accomplished by genome shuffling.

In certain aspects of these embodiments step (i) is repeated one or more times, first on the parent and then on subsequent mutants, prior to the measuring and selection of mutants in steps (ii) and (iii).

In yet another embodiment, step (iv) is followed by further screening of selected mutants by measuring plant growth promotion abilities of such mutants and selecting mutants with improved plant growth promotion abilities compared to the parent strain or other predecessor mutants. Mutants having improved plant growth promotion abilities compared to the parent strain may be further mutagenized and selected for enhanced production of the bacterial compounds described above and/or further improved plant growth promotion capabilities.

The present invention also provides a method for improving plant growth promotion, e.g., by increasing plant growth or yield, by applying superoxide dismutase to a plant, plant part or the locus surrounding the plant. In one embodiment, the superoxide dismutase is purified or partially purified. In another embodiment, it is applied in a fermentation product of a bacteria capable of producing SOD.

### DETAILED DESCRIPTION

Bacillus firmus I-1582 is described in U.S. Pat. No. 6,406,690, and in several other patent applications. For example, U.S. Patent Application Publication No. 2011/0110906, describes combinations of strain I-1582 with insecticides and fungicides and methods of using such combinations to improve overall plant vigor and yield. In addition, U.S. Patent Application Publication No. US 2011/0154544, describes treatment of genetically modified seed with this strain to improve overall plant vigor and yield. Bacillus firmus I-1582 is a bacterial strain with nematode control properties and the ability to colonize plant root systems. It was deposited with the Collection Nationale de Cultures de Microorganismes (CNCM), Institute Pasteur, France, on May 29, 1995, and was assigned Accession No. CNCM I-1582.

The present invention provides methods for generating mutants of Bacillus firmus I-1582 with enhanced plant growth-promotion and for screening and development of such mutants. In one embodiment, the method includes generating mutants and then screening such mutants for enhanced plant growth promotion in comparison to the parent strain. In one embodiment, plant, crop, fruit or vegetable yield or plant mass is increased by about 1% to about 10%, by about 2% to about 15%, by about 2% to about 20% compared to an untreated plant, crop, fruit, or vegetable. In yet another aspect, plant, crop, fruit or vegetable yield is increased by about 1%, about 2%, about 3%, about 4%, about 5%, about 10%, about 20%, about 30%, compared to an untreated plant, crop, fruit, or vegetable or compared to a plant, crop, fruit or vegetable treated with the wildtype strain.

In another embodiment the mutants are further screened for nematode control that is at least as effective as that of the parent strain. Nematode control may be tested as described in U.S. Pat. No. 9,554,578, using an appropriate amount of whole broth culture of the Bacillus firmus strains described in this application. Methods for preparing whole broth culture of the herein-described Bacillus firmus strains are provided in the Examples. Other methods for screening for nematode control are well-known to those of skill in the art.

In another embodiment the mutants are screened for production of indole acetic acid, or IAA, the chemical structure of which is shown below.

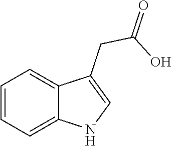 [SMILES: O=C(O)Cc1c[nH]c2ccccc12]

Plants use phytohormones, such as auxins, including IAA, to influence cellular function. Some studies have shown a positive correlation between microbial IAA production and plant growth. See Shahab, S., et al., “Indole Acetic Acid Production and Enhanced Plant Growth Promotion by Indigenous PSBs,” African Journal of Agricultural Research, Vol. 4 (11), (November 2009), 1312-1316; see also Marques, A., et al., “Assessment of the Plant Growth Promotion Abilities of Six Bacterial Isolates Using Zea mays as Indicator Plant,” Soil Biology and Biochemistry, 42 (2010), 1229-1235. In one particular aspect of this embodiment, increased production of IAA is used as a primary screen of mutants. In another aspect, IAA production and enhanced plant growth promotion are both required in the mutants.

In one aspect of this embodiment, IAA production of the mutant strain is increased by at least one fold, by at least two fold, by at least three fold, by at least four fold, by at least five fold, by at least six fold, by at least seven fold, by at least eight fold, by at least nine fold, or by at least ten fold over IAA production in the parent strain or strains.

Plants produce active oxygen species as an carly response to pathogen infection. Superoxide dismutase (“SOD”) acts as an antioxidant and protects cellular components from being oxidized by reactive oxygen species. Various publications have reported an increase in SOD production by plants treated with plant growth promoting bacteria. See, for example, Genthilraja, G., et al., “Plant Growth Promoting Rhizobacteria (PGPR) and Entomopathogenic Fungus Bioformulation Enhance The Expression of Defense Enzymes and Pathogenesis-Related Proteins in Groundnut Plants Against Leafminer Insect and Collar Rot Pathogen,” Physiological and Molecular Plant Pathology (2013) 82:10-19. In addition, Applicant conducted a study showing that SOD applied directly to soil in which corn seeds were planted increased corn shoot weight by 12% over the untreated control. Briefly, corn seeds were planted in potting mix in cell trays and the soil treated two times during the study with 2 mL of a 0.6 U/mL solution of SOD, with two replicates per treatment. SOD was obtained from Sigma Aldrich (Product Number S9697, Cas Number 9054-89-1, superoxide dismutase bovine-recombinant, expressed in E. coli). Seeds were grown for fourteen days after which shoot weight of the untreated control and treated plants was measured.

Therefore, in yet another aspect of this embodiment of the invention, the mutants are further screened for superoxide dismutase activity that is higher than that of the parent strain. In one aspect of this embodiment, superoxide dismutase activity is increased by at least one fold, by at least two fold, by at least three fold, by at least four fold, by at least five fold, by at least six fold, by at least seven fold, by at least eight fold, by at least nine fold, or by at least ten fold over superoxide dismutase activity of the parent strain or strains.

Some Bacillus firmus strains, such as Bacillus firmus I-1582, produce protease proteins, which are known to have nematicidal activity. See, for example, Geng, C., “A Novel Serine Protease, Sep1, from Bacillus firmus DS-1 Has Nematicidal Activity and Degrades Multiple Intestinal-Associated Nematode Proteins,” Scientific Reports, published online on Apr. 27, 2016, at www.nature.com/scientificreports. Therefore, in yet another aspect of the methods of this invention, the mutants are further screened for protease activity that is at least as high as that of the parent strain. In one aspect of this embodiment, protease activity is increased by at least one fold, by at least two fold, by at least three fold, by at least four fold, by at least five fold, by at least six fold, by at least seven fold, by at least eight fold, by at least nine fold, or by at least ten fold over protease activity of the parent strain or strains.

The term “mutant” refers to a genetic variant derived from a Bacillus firmus strain. In one embodiment, the mutant has one or more or all the identifying (functional) characteristics of Bacillus firmus strain I-1582, of Bacillus firmus strain NRRL B-67003, or of Bacillus firmus strain NRRL B-67518. In a particular instance, the mutant or a fermentation product thereof enhances plant health, including yield. Such mutants may be genetic variants having a genomic sequence that has greater than about 85%, greater than about 90%, greater than about 95%, greater than about 98%, or greater than about 99% sequence identity to one or more of the above-referenced Bacillus firmus strains. Mutants may be obtained by treating cells of Bacillus firmus strain I-1582, NRRL B-67003 or NRRL B-67518 with chemicals or irradiation or by selecting spontaneous mutants from a population of such Bacillus firmus strain cells (such as phage resistant or antibiotic resistant mutants), by genome shuffling, as described below, or by other means well known to those practiced in the art.

Genome shuffling among Bacillus strains can be facilitated through the use of a process called protoplast fusion. The process begins with the formation of protoplasts from vegetative bacillary cells. The removal of peptidoglycan cell wall, typically using lysozyme and an osmotic stabilizer, results in the formation of a protoplast. This process is visible under a light microscope with the appearance of spherical cells. Addition of PEG, polyethylene glycol, then induces fusion among protoplasts, allowing genetic contents of two or more cells to come in contact facilitating recombination and genome shuffling. Fused cells then repartition and are recovered on a solid growth medium. During recovery, protoplasts rebuild peptidoglycan cell walls, transitioning back to bacillary shape. See Schaeffer, et. al., (1976) PNAS USA, vol. 73, 6:2151-2155).

Specific examples of generating Bacillus firmus mutants are described below in the Examples section.

The mutant strain can be any mutant strain that has one or more or all the identifying characteristics of Bacillus firmus strain I-1582, Bacillus firmus strain NRRL B-67003, and/or Bacillus firmus strain NRRL B-67518 and, in particular, plant growth-promoting activity that is better than that of one or more of such strains. In another embodiment, the mutant strain has nematode control activity that is at least as effective as that of the parent strain. In yet another embodiment the mutant produces more indole acetic acid (“IAA”) than does the parent strain.

The term “plant growth promotion” or “plant growth-promoting,” as used herein, refers to the ability of a microorganism to exert a beneficial effect on plant growth or crop yield. For example, this may relate to increased length and/or fresh and/or dry weights of roots and shoots of treated plants or crops compared to untreated plants or crops. Other indications of a beneficial effect on plant growth include enhanced nodulation in soybeans and increased number of branching roots.

Plant growth promotion may also be characterized by improved plant vigor, including the following: (a) improved vitality of the plant, (b) improved quality of the plant and/or of the plant products, e.g., enhanced protein content, (c) improved visual appearance, (d) delay of senescence, (e) enhanced root growth and/or more developed root system (e.g., determined by the dry mass of the root), (f) enhanced nodulation, in particular rhizobial nodulation, (g) longer panicles, (h) bigger leaf blade, (i) less dead basal leaves, (j) increased chlorophyll content, (k) prolonged photosynthetically active period, (1) increased or improved plant stand density, (m) less plant verse (lodging), (n) increased plant weight, (o) increased plant height, (p) tillering increase, (q) stronger and/or more productive tillers, (r) less non-productive tillers, (s) enhanced photosynthetic activity and/or enhanced pigment content and thus greener leaf color, (t) earlier and/or improved germination, (u) improved and/or more uniform and/or earlier emergence, (v) increased shoot growth, (w) earlier flowering, (x) earlier fruiting, (y) earlier grain maturity, (2) less fertilizers needed, (aa) less seeds needed.

According to the present invention, “increased yield” of a plant, in particular of an agricultural, silvicultural and/or ornamental plant means that the yield of a product of the respective plant is increased by a measurable amount over the yield of the same product of the plant produced under the same conditions, but without the application of the composition of the invention or with the application of a parent bacterial strain, such as Bacillus firmus I-1582. According to the present invention, it is preferred that the yield be increased by at least 0.5%, or by at least 1%, or by at least 2%, or by at least 4%, or by at least 5%, or by at least 10% when compared to appropriate controls.

Plant growth promotion ability refers to the ability of a strain to improve one of the above properties of a plant after application to a plant, plant part or to the locus of a plant compared to a plant that has not been treated with the plant growth promotion strain. In one embodiment, the strains of the present invention increase yield or total plant weight by at least about 0.5%, or by at least about 1%, or by at least about 2%, or by at least about 3%, or by at least about 5%, or by at least about 6%, or by at least about 7%, or by at least about 8%, or by at least about 9%, or by at least about 10%, or by at least about 11%, or by at least about 12% when compared to plants produced under the same conditions but without treatment by a plant growth promoting strain.

Mutants of Bacillus spp., including Bacillus subtilis, Bacillus amyloliquefaciens, Bacillus pumilus, and Bacillus firmus may be generated using spontancous mutagenesis, mutagenesis induced by chemicals or irradiation, genome shuffling or a combination of such techniques. Such mutants may then be screened for improved production of various plant growth regulators, such as IAA, gibberellin, or cytokinin; improved production or activity of 1-aminocyclopropane-1-carboxylate (ACC) deaminase or superoxide dismutase; improved production of bacterial compounds that contribute to induced systemic resistance in plants, such as siderophores, salicylic acid and lipopolysaccharides; and/or for enhanced ability to promote plant growth promotion compared to parent strains. Multiple rounds of mutagenesis, with and without screening between rounds, may be used to generate and screen mutants. Fermentation products of mutants having one or more improved attributes may be produced and applied to plants to promote plant growth.

In a method according to the invention a composition containing Bacillus firmus 1-1582, Bacillus firmus strain NRRL B-67003, Bacillus firmus NRRL B-67518 or a plant-growth-promoting mutant of any of the aforementioned strains can be applied to any plant or any part of any plant grown in any type of media used to grow plants (e.g., soil, vermiculite, shredded cardboard, and water) or applied to plants or the parts of plants grown acrially, such as orchids or staghorn ferns. The composition may for instance be applied by spraying, atomizing, vaporizing, scattering, dusting, watering, squirting, sprinkling, pouring or fumigating. As already indicated above, application may be carried out at any desired location where the plant of interest is positioned, such as agricultural, horticultural, forest, plantation, orchard, nursery, organically grown crops, turfgrass and urban environments.

The strains and compositions of the present invention can be applied to the seed, plant, or plant parts either as a powder, aqueous or non-aqueous solution. Powders can be cither dry, wettable powders, or water dispersable granules. In some embodiments, the spore-forming bacterium is a solution, emulsifiable concentrate, wettable powder, suspension concentrate, soluble powder, granules, suspension-emulsion concentrate, natural and synthetic materials impregnated with active compounds and fine control release capsules. The strains and compositions of the present invention in a liquid or dry form may be admixed with the soil prior to, at the time of, or after planting. In one embodiment, the composition is in a liquid state admixed with the soil prior to or at the time of planting.

Compositions of the present invention include biologically pure cultures of the strains described herein. The term “biologically pure culture” refers to a population of cells growing in the absence of other species in a predetermined culture media under controlled laboratory or manufacturing conditions. Biologically pure cultures of mutants of Bacillus firmus I-1582, including Bacillus firmus NRRL B-67003, Bacillus firmus NRRL B-67518 and mutants derived therefrom, may be obtained according to methods well known in the art, including by using the media and other methods described in the examples below or in U.S. Pat. No. 6,406,690.

Conventional large-scale microbial culture processes include submerged fermentation, solid state fermentation, or liquid surface culture. During the fermentation, as nutrients are depleted, cells begin the transition from growth phase to sporulation phase, such that the final product of fermentation is largely spores, metabolites and residual fermentation medium. Sporulation is part of the natural life cycle of Bacillus firmus and is generally initiated by the cell in response to nutrient limitation. Fermentation is configured to obtain high levels of colony forming units and to promote sporulation. The bacterial cells, spores and metabolites in culture media resulting from fermentation may be used directly or concentrated by conventional industrial methods, such as centrifugation or filtration such as tangential-flow filtration or depth filtration, and evaporation.

Compositions of the present invention include the products of the microbial culture processes described herein. In embodiments in which submerged fermentation is used as the culture process, the product is referred to as a “fermentation broth.” Such broth may be concentrated, as described above. The concentrated fermentation broth may be washed, for example, via a diafiltration process, to remove residual fermentation broth and metabolites. The term “broth concentrate,” as used herein, refers to fermentation broth that has been concentrated by conventional industrial methods, as described above, but remains in liquid form. The term “fermentation product,” as used herein, refers to fermentation broth, broth concentrate and/or dried fermentation broth or broth concentrate.

The fermentation broth or broth concentrate can be dried with or without the addition of carriers using conventional drying processes or methods such as spray drying, freeze drying, tray drying, fluidized-bed drying, drum drying, or evaporation.

The resulting dry products may be further processed, such as by milling or granulation, to achieve a specific particle size or physical format. Carriers, described below, may also be added post-drying.

The term “fermentation product,” as used herein, refers to fermentation broth, broth concentrate and/or dried fermentation broth or broth concentrate, referred to herein as dried fermentation broth.

Cell-free preparations of fermentation broth of the strains of the present invention can be obtained by any means known in the art, such as extraction, centrifugation and/or filtration of fermentation broth. Those of skill in the art will appreciate that so-called cell-free preparations may not be devoid of cells but rather are largely cell-free or essentially cell-free, depending on the technique used (e.g., speed of centrifugation) to remove the cells. The resulting cell-free preparation may be dried and/or formulated with components that aid in its application to plants or to plant growth media. Concentration methods and drying techniques described above for fermentation broth are also applicable to cell-free preparations.

In one embodiment, the fermentation product comprises at least about 1×10⁵ colony forming units (CFU) of the microorganism (e.g., Bacillus firmus strain NRRL B-67003, Bacillus firmus NRRL B-67518, or a plant health enhancing or plant growth promoting mutant strain thereof)/mL broth. In another embodiment, the fermentation product comprises at least about 1 x 106 CFU of the microorganism (e.g., Bacillus firmus strain NRRL B-67003, Bacillus firmus NRRL B-67518, or a plant growth-promoting mutant strain thereof)/mL broth. In yet another embodiment, the fermentation product comprises at least about 1×10⁷ CFU of the microorganism (e.g., Bacillus firmus strain NRRL B-67003, Bacillus firmus NRRL B-67518, or a plant growth-promoting mutant strain thereof)/mL broth. In another embodiment, the fermentation product comprises at least about 1×10⁸ CFU of the microorganism (e.g., Bacillus firmus strain NRRL B-67003, Bacillus firmus NRRL B-67518, or a plant growth-promoting mutant strain thereof)/mL broth. In another embodiment, the fermentation product comprises at least about 1×10⁹ CFU of the microorganism (e.g., Bacillus firmus strain NRRL B-67003, Bacillus firmus NRRL B-67518, or a plant growth-promoting mutant strain thereof)/mL broth. In another embodiment, the fermentation product comprises at least about 1×10¹⁰ CFU of the microorganism (e.g., Bacillus firmus strain NRRL B-67003, Bacillus firmus NRRL B-67518, or a plant growth-promoting mutant strain thereof)/mL broth. In another embodiment, the fermentation product comprises at least about 1×10¹¹ CFU of the microorganism (e.g., Bacillus firmus strain NRRL B-67003, Bacillus firmus NRRL B-67518, or a plant growth-promoting mutant strain thereof)/mL broth.

In another embodiment the fermentation product is a broth concentrate or a dried broth that comprises at least about 1×10⁸ colony forming units (CFU) of the microorganism (e.g., Bacillus firmus strain NRRL B-67003, Bacillus firmus NRRL B-67518, or a plant health enhancing mutant strain thereof)/mL broth. In another embodiment the fermentation product is a broth concentrate or a dried fermentation broth that comprises at least about 1×10⁹ CFU of the microorganism (e.g., Bacillus firmus strain NRRL B-67003, Bacillus firmus NRRL B-67518, or a plant health enhancing mutant strain thereof)/mL broth. In another embodiment the fermentation product is a broth concentrate or a dried fermentation broth that comprises at least about 1×10¹⁰ CFU of the microorganism (e.g., Bacillus firmus strain NRRL B-67003, Bacillus firmus NRRL B-67518, or a plant health enhancing mutant strain thereof)/mL broth. In another embodiment the fermentation product is a broth concentrate or a dried fermentation broth that comprises at least about 1×10¹¹ CFU of the microorganism (e.g., Bacillus firmus strain NRRL B-67003, Bacillus firmus NRRL B-67518, or a plant health enhancing mutant strain thereof)/mL broth. In another embodiment the fermentation product is a broth concentrate or a dried fermentation broth that comprises at least about 1×10¹² CFU of the microorganism (e.g., Bacillus firmus strain NRRL B-67003, Bacillus firmus NRRL B-67518, or a plant health enhancing mutant strain thereof)/mL broth. In another embodiment the fermentation product is a broth concentrate or a dried fermentation broth that comprises about 1×10⁸ CFU to about 1×10¹² CFU of the microorganism (e.g., Bacillus firmus strain NRRL B-67003, Bacillus firmus NRRL B-67518, or a plant health enhancing mutant strain thereof)/mL broth. In another embodiment the fermentation product is a broth concentrate or a dried fermentation broth that comprises about 1×10¹⁰ CFU to about 1×10¹¹ CFU of the microorganism (e.g., Bacillus firmus strain NRRL B-67003, Bacillus firmus NRRL B-67518, or a plant health enhancing mutant strain thereof)/mL broth.

In another embodiment, the fermentation product comprises about 1%, about 2%, about 3%, about 4%, about 5%, about 6%, about 7%, about 8%, about 9%, about 10%, about 15%, about 20%, or about 25%, fermentation solids. Fermentation solids include spores, vegetative cells and unspent fermentation media. In certain aspects, the fermentation product comprises about 1% to about 60% fermentation solid, e.g., any range within 1% to 60%, such as 1% to 50%, 1% to 40%, 1% to 30%, 1% to 25%, 1% to 20%, 1% to 15%, 1% to 10%, 30% to 60%, 40% to 60%, etc. In certain aspects, the fermentation product comprises about 1% to about 25% fermentation solids, about 1% to about 20% fermentation solids, about 1% to about 15% fermentation solids, or about 1% to about 10% fermentation solids.

The inventive compositions can be used as such or, depending on their particular physical and/or chemical properties, in the form of their formulations or the use forms prepared therefrom, such as acrosols, capsule suspensions, cold-fogging concentrates, warm-fogging concentrates, encapsulated granules, fine granules, flowable concentrates for the treatment of seed, ready-to-use solutions, dustable powders, emulsifiable concentrates, oil-in-water emulsions, water-in-oil emulsions, macrogranules, microgranules, oil-dispersible powders, oil-miscible flowable concentrates, oil-miscible liquids, gas (under pressure), gas generating product, foams, pastes, pesticide coated seed, suspension concentrates, oil dispersion, suspoemulsion concentrates, soluble concentrates, suspensions, including encapsulated suspensions, where, for example, an oil dispersion containing solid particles is encapsulated in water, wettable powders, soluble powders, dusts and granules, water-soluble and water-dispersible granules or tablets, water-soluble and water-dispersible powders for the treatment of seed, wettable powders, natural products and synthetic substances impregnated with active ingredient, microdispersions, and also microencapsulations in polymeric substances and in coating materials for seed, and also ULV cold-fogging and warm-fogging formulations.

In a certain aspect the biologically pure cultures or related fermentation products of the present invention are combined with an agriculturally acceptable carrier. Such agriculturally acceptable carriers may be the formulation inerts described below.

In a certain aspect the compositions of the present invention are formulated for seed treatment as dry dustable powders, flowable suspensions or suspension concentrates, liquid solutions, water soluble powers or water-dispersible powers.

In some embodiments, the inventive compositions are liquid formulations. Non-limiting examples of liquid formulations include suspension concentrations and oil dispersions. In other embodiments, the inventive compositions are solid formulations. Non-limiting examples of liquid formulations include freeze-dried powders and spray-dried powders.

Compositions of the present invention may include formulation inerts added to compositions comprising cells, cell-free preparations or metabolites to improve efficacy, stability, and usability and/or to facilitate processing, packaging and end-use application in agriculture. Such formulation inerts and ingredients may include carriers, stabilization agents, nutrients, or physical property modifying agents, which may be added individually or in combination. In some embodiments, the carriers may include liquid materials such as water, oil, and other organic or inorganic solvents and solid materials such as minerals, polymers, or polymer complexes derived biologically or by chemical synthesis. In some embodiments, the carrier is a binder or adhesive that facilitates adherence of the composition to a plant part, such as a seed or root. See, for example, Taylor, A. G., et al., “Concepts and Technologies of Selected Seed Treatments,” Annu. Rev. Phytopathol. 28:321-339 (1990). The stabilization agents may include anti-caking agents, anti-oxidation agents, desiccants, protectants or preservatives. The nutrients may include carbon, nitrogen, and phosphors sources such as sugars, polysaccharides, oil, proteins, amino acids, fatty acids and phosphates. The physical property modifiers may include bulking agents, wetting agents, thickeners, pH modifiers, rheology modifiers, dispersants, adjuvants, surfactants, antifreeze agents or colorants. In some embodiments, the composition comprising cells, cell-free preparation or metabolites produced by fermentation can be used directly with or without water as the diluent without any other formulation preparation.

In some embodiments, the formulation inerts are added after concentrating fermentation broth and during and/or after drying.

All plants and plant parts can be treated in accordance with the invention. In the present context, plants are understood as meaning all plants and plant populations, such as desired and undesired wild plants or crop plants (including naturally occurring crop plants). Crop plants can be plants which can be obtained by traditional breeding and optimization methods or by biotechnological and recombinant methods, or combinations of these methods, including the transgenic plants and including the plant varieties capable or not of being protected by Plant Breeders' Rights. Plant parts are understood as meaning all aerial and subterranean parts and organs of the plants, such as shoot, leaf, flower and root, examples which may be mentioned being leaves, needles, stalks, stems, flowers, fruiting bodies, fruits and seeds, and also roots, tubers and rhizomes. The plant parts also include crop material and vegetative and generative propagation material, for example cuttings, tubers, rhizomes, slips and seeds.

As has already been mentioned above, all plants and their parts may be treated in accordance with the invention. In a preferred embodiment, plant species and plant varieties, and their parts, which grow wild or which are obtained by traditional biological breeding methods such as hybridization or protoplast fusion are treated. In a further preferred embodiment, transgenic plants and plant varieties which have been obtained by recombinant methods, if appropriate in combination with traditional methods (genetically modified organisms), and their parts are treated. The term “parts” or “parts of plants” or “plant parts” has been explained hereinabove. Plants of the plant varieties which are in each case commercially available or in use are especially preferably treated in accordance with the invention. Plant varieties are understood as meaning plants with novel traits which have been bred both by traditional breeding, by mutagenesis or by recombinant DNA techniques. They may take the form of varieties, races, biotypes and genotypes.

The treatment of the plants and plant parts with the compositions according to the invention is carried out directly or by acting on the environment, habitat or storage space using customary treatment methods, for example by dipping, spraying, atomizing, misting, evaporating, dusting, fogging, scattering, foaming, painting on, spreading, injecting, drenching, trickle irrigation and, in the case of propagation material, in particular in the case of seed, furthermore by the dry seed treatment method, the wet seed treatment method, the slurry treatment method, by encrusting, by coating with one or more coats and the like. It is furthermore possible to apply the active substances by the ultra-low volume method or to inject the active substance preparation or the active substance itself into the soil.

Preferred plants are those from the group of the useful plants, ornamentals, turfs, generally used trees which are employed as ornamentals in the public and domestic sectors, and forestry trees. Forestry trees comprise trees for the production of timber, cellulose, paper and products made from parts of the trees.

The term “useful plants” as used in the present context refers to crop plants which are employed as plants for obtaining foodstuffs, feedstuffs, fuels or for industrial purposes.

The useful plants which can be treated and/or improved with the compositions and methods of the present invention include for example the following types of plants: turf, vines, cereals, for example wheat, barley, rye, oats, rice, maize and millet/sorghum; beet, for example sugar beet and fodder beet; fruits, for example pome fruit, stone fruit and soft fruit, for example apples, pears, plums, peaches, almonds, cherries and berries, for example strawberries, raspberries, blackberries; legumes, for example beans, lentils, peas and soybeans; oil crops, for example oilseed rape, mustard, poppies, olives, sunflowers, coconuts, castor oil plants, cacao and peanuts; cucurbits, for example pumpkin/squash, cucumbers and melons; fibre plants, for example cotton, flax, hemp and jute; citrus fruit, for example oranges, lemons, grapefruit and tangerines; vegetables, for example spinach, lettuce, asparagus, cabbage species, carrots, onions, tomatoes, potatoes and bell peppers; Lauraceae, for example avocado, Cinnamomum, camphor, or else plants such as tobacco, nuts, coffee, aubergine, sugar cane, tea, pepper, grapevines, hops, bananas, latex plants and ornamentals, for example flowers, shrubs, deciduous trees and coniferous trees.

The present invention can also be applied to any turf grasses, including cool-season turf grasses and warm-season turf grasses. Examples of cold-season turf grasses are bluegrasses (Poa spp.), such as Kentucky bluegrass (Poa pratensis L.), rough bluegrass (Poa trivialis L.), Canada bluegrass (Poa compressa L.), annual bluegrass (Poa annua L.), upland bluegrass (Poa glaucantha Gaudin), wood bluegrass (Poa nemoralis L.) and bulbous bluegrass (Poa bulbosa L.); bentgrasses (Agrostis spp.) such as creeping bentgrass (Agrostis palustris Huds.), colonial bentgrass (Agrostis tenuis Sibth.), velvet bentgrass (Agrostis canina L.), South German mixed bentgrass (Agrostis spp. including Agrostis tenuis Sibth., Agrostis canina L., and Agrostis palustris Huds.), and redtop (Agrostis alba L.);
fescues (Festuca spp.), such as red fescue (Festuca rubra L. spp. rubra), creeping fescue (Festuca rubra L.), chewings fescue (Festuca rubra commutata Gaud.), sheep fescue (Festuca ovina L.), hard fescue (Festuca longifolia Thuill.), hair fescue (Festucu capillata Lam.), tall fescue (Festuca arundinacea Schreb.) and meadow fescue (Festuca elanor L.);
ryegrasses (Lolium spp.), such as annual ryegrass (Lolium multiflorum Lam.), perennial ryegrass (Lolium perenne L.) and Italian ryegrass (Lolium multiflorum Lam.);
and wheatgrasses (Agropyron spp.), such as fairway wheatgrass (Agropyron cristatum (L.) Gaertn.), crested wheatgrass (Agropyron desertorum (Fisch.) Schult.) and western wheatgrass (Agropyron smithii Rydb.).

Examples of further cool-season turf grasses are beachgrass (Ammophila breviligulata Fern.), smooth bromegrass (Bromus inermis Leyss.), cattails such as timothy (Phleum pratense L.), sand cattail (Phleum subulatum L.), orchardgrass (Dactylis glomerata L.), weeping alkaligrass (Puccinellia distans (L.) Parl.) and crested dog's-tail (Cynosurus cristatus L.).

Examples of warm-season turf grasses are Bermuda grass (Cynodon spp. L. C. Rich), zoysia grass (Zoysia spp. Willd.), St. Augustine grass (Stenotaphrum secundatum Walt Kuntze), centipede grass (Eremochloa ophiuroides Munro Hack.), carpetgrass (Axonopus affinis Chase), Bahia grass (Paspalum notatum Flugge), Kikuyu grass (Pennisetum clandestinum Hochst. ex Chiov.), buffalo grass (Buchloe dactyloids (Nutt.) Engelm.), blue grama (Bouteloua gracilis (H. B. K.) Lag. ex Griffiths), seashore paspalum (Paspalum vaginatum Swartz) and sidcoats grama (Bouteloua curtipendula (Michx. Torr.). Cool-season turf grasses are generally preferred for the use according to the invention. Especially preferred are bluegrass, benchgrass and redtop, fescues and ryegrasses. Bentgrass is especially preferred.

In certain aspects, the compositions of the present invention are applied to seed at about 1×10⁵ to about 1×10⁸ colony forming units (CFU) of plant growth-promoting mutant of Bacillus firmus strain I-1582, Bacillus firmus strain NRRL B-67003, Bacillus firmus NRRL B-67518, or plant growth-promoting mutant strain derived therefrom per seed, depending on seed size. For example, for a corn seed, the application rate is about 1×10⁶ CFU/seed to about 1×10⁷ CFU/seed, and for soy the application rate is about 1×10CFU/seed to about 1×10⁶ CFU/seed.

When used as a soil treatment, the compositions and spore-forming bacterial cells of the present invention can be applied as a soil surface drench, shanked-in, injected and/or applied in-furrow or by mixture with irrigation water. The rate of application for drench soil at any stage of plant growth is about 1×10¹³ to about 1×10¹⁵ colony forming units (CFU) of plant growth-promoting mutant of Bacillus firmus strain I-1582, plant growth-promoting Bacillus firmus strain NRRL B-67003, Bacillus firmus NRRL B-67518, or plant growth-promoting mutant strain derived therefrom per hectare. In other aspects, the compositions of the present invention are applied at about 1×10¹⁴ to about 1×10¹⁵ colony forming units (CFU) of plant growth-promoting Bacillus firmus strain NRRL B-67003, Bacillus firmus NRRL B-67518, or plant growth-promoting mutant strain derived therefrom per hectare. In yet other aspects, the compositions of the present invention are applied at about 1×10¹⁴ to about 5×10¹⁴ colony forming units (CFU) of plant growth-promoting mutant of Bacillus firmus strain I-1582, plant growth-promoting Bacillus firmus strain NRRL B-67003, Bacillus firmus NRRL B-67518, or plant growth-promoting mutant strain derived therefrom per hectare.

The application rate for foliar applications, such as applications to turf, are the same as those disclosed above for soil treatment.

In certain aspects, the fermentation product further comprises a formulation ingredient. The formulation ingredient may be a wetting agent, extender, solvent, spontancity promoter, emulsifier, dispersant, frost protectant, thickener, and/or an adjuvant. In one embodiment, the formulation ingredient is a wetting agent.

Compositions of the present invention may include formulation ingredients added to compositions of the present invention to improve recovery, efficacy, or physical properties and/or to aid in processing, packaging and administration. Such formulation ingredients may be added individually or in combination.

The formulation ingredients may be added to compositions comprising cells, cell-free preparations and/or metabolites to improve efficacy, stability, and physical properties, usability and/or to facilitate processing, packaging and end-use application. Such formulation ingredients may include carriers, inerts, stabilization agents, preservatives, nutrients, or physical property modifying agents, which may be added individually or in combination. In some embodiments, the carriers may include liquid materials such as water, oil, and other organic or inorganic solvents and solid materials such as minerals, polymers, or polymer complexes derived biologically or by chemical synthesis. In some embodiments, the formulation ingredient is a binder, adjuvant, or adhesive that facilitates adherence of the composition to a plant part, such as leaves, seeds, or roots. See, for example, Taylor, A. G., et al., “Concepts and Technologies of Selected Seed Treatments,” Annu. Rev. Phytopathol., 28:321-339 (1990). The stabilization agents may include anti-caking agents, anti-oxidation agents, anti-settling agents, antifoaming agents, desiccants, protectants or preservatives. The nutrients may include carbon, nitrogen, and phosphorus sources such as sugars, polysaccharides, oil, proteins, amino acids, fatty acids and phosphates. The physical property modifiers may include bulking agents, wetting agents, thickeners, pH modifiers, rheology modifiers, dispersants, adjuvants, surfactants, film-formers, hydrotropes, builders, antifreeze agents or colorants. In some embodiments, the composition comprising cells, cell-free preparation and/or metabolites produced by fermentation can be used directly with or without water as the diluent without any other formulation preparation. In a particular embodiment, a wetting agent, or a dispersant, is added to a dried broth concentrate, such as a freeze-dried or spray-dried powder. A wetting agent increases the spreading and penetrating properties, or a dispersant increases the dispersibility and solubility of the active ingredient (once diluted) when it is applied to surfaces. Exemplary wetting agents are known to those of skill in the art and include sulfosuccinates and derivatives, such as MULTIWET™ MO-70R (Croda Inc., Edison, NJ); siloxanes such as BREAK-THRU® (Evonik, Germany); nonionic compounds, such as ATLOX™ 4894 (Croda Inc., Edison, NJ); alkyl polyglucosides, such as TERWET® 3001 (Huntsman International LLC, The Woodlands, Texas); C12-C14 alcohol ethoxylate, such as TERGITOL® 15-S-15 (The Dow Chemical Company, Midland, Michigan); phosphate esters, such as RHODAFAC® BG-510 (Rhodia, Inc.); and alkyl ether carboxylates, such as EMULSOGEN™ LS (Clariant Corporation, North Carolina).

### DEPOSIT INFORMATION

A sample of a Bacillus firmus strain of the invention has been deposited with the Agricultural Research Service Culture Collection located at the National Center for Agricultural Utilization Research, Agricultural Research Service, U.S. Department of Agriculture, 1815 North University Street, Peoria, Illinois 61604, U.S.A., under the Budapest Treaty on Oct. 24, 2017, and has been assigned the following depository designation: NRRL B-67518.

A sample of a Bacillus firmus strain of the invention has been deposited with the Agricultural Research Service Culture Collection located at the National Center for Agricultural Utilization Research, Agricultural Research Service, U.S. Department of Agriculture, 1815 North University Street, Peoria, Illinois 61604, U.S.A., under the Budapest Treaty on Mar. 18, 2015, and has been assigned the following depository designation: NRRL B-67003.

A sample of Bacillus firmus I-1582 (products known as BIONEM™, VOTIVO®, FLOCTER®), disclosed in U.S. Pat. No. 6,406,690 (which is herein incorporated by reference) was deposited with the CNCM on May 29, 1995, with Accession No. CNCM I-1582. CNCM is the abbreviation for the Collection Nationale de Cultures de Microorganismes, Institute Pasteur, France, having the address of Institut Pasteur, 25 Rue du Docteur Roux, F-75724 Paris Cedex 15, France.

All strains described herein and having an accession number in which the prefix is NRRL or CNCM have been deposited with the above-described respective depositary institution in accordance with the Budapest Treaty on the International Recognition of the Deposit of Microorganisms for the Purposes of Patent Procedure.

The Bacillus firmus strain has been deposited under conditions that assure that access to the culture will be available during the pendency of this patent application to one determined by the Commissioner of Patents and Trademarks to be entitled thereto under 37 C.F.R. § 1.14 and 35 U.S.C. § 122. The deposit represents a substantially pure culture of the deposited Bacillus firmus strain. The deposit is available as required by foreign patent laws in countries wherein counterparts of the subject application or its progeny are filed. However, it should be understood that the availability of a deposit does not constitute a license to practice the subject invention in derogation of patent rights granted by governmental action.

The following examples are given for purely illustrative and non-limiting purposes of the present invention.

### EXAMPLES

### Example 1

### Random Mutagenesis to Generate Improved Mutants

With the goal of generating mutants with improved ability to enhance plant health, mutants were created from the parent strains, Bacillus firmus I-1582 and variants thereof. The parent strains were subjected to mutagenesis using either N-methyl-N′-nitro-N-nitrosoguanidine (“NTG”) or Ethyl Methanesulfonate (“EMS”). NTG and EMS treatments were used at concentrations suitable to yield acceptable kill percentages of actively dividing Bacillus firmus. Kill percentages were assessed through CFU analysis of pre-and post-treatment samples. Based on screening results certain isolates or variant pools were mutagenized further, in the same manner as described above.

### Example 2

### Screening for Increased IAA Production

Mutants generated after the first or second round of mutagenesis were screened for enhanced production of indole acetic acid (“IAA”) in a high throughput manner. Initial screens were conducted in 96 deep well seed blocks of Luria Broth followed by screening in 48-deep well production blocks containing a high protein fermentation media inoculated with the parent strains or with the mutant strains. Production blocks were incubated at 37° C. and 220 rpm for 3 days.

The whole broth from each well of the production block was tested for IAA production as follows using ultra-high pressure liquid chromatography (UPLC) and mass spectrometry. The production block was centrifuged. 0.5 mL supernatant was transferred to a 3 KDa MWCO filter block secure atop a receiving block. This combination of filtration and receiving blocks was centrifuged to collect the filtrate. The filter block was removed and then the receiving block was then assayed for IAA content using UPLC chromatography and mass spectrometry. Specifically, a sample was injected onto a Waters Acquity HSS T3 column (100 Å, 1.8 μm, 50×2.1 mm) fitted with a HSS T3 guard column. The column was eluted with a 3 minute acetonitrile/water gradient (see below). The flow rate was 0.6 mL/min. IAA was detected via mass spectrometry ESI—single ion mode with a mass over charge (m/z) of 174.1 for IAA. IAA elutes as a single peak with an approximate retention time of 1 minute.

Top isolates were selected for a confirmation screen (similar to as previously described but each isolate was assayed in replicates of 4 or 8). Bacillus firmus NRRL B-67003 showed a 1.3 fold increase compared to the parent strain. The vast majority of mutants screened did not show an increase in IAA production compared to the parent strain.

### Example 3

### Biological Efficacy of B. firmus NRRL B-67003

Cell tray assays and greenhouse tests were conducted to compare plant growth promotion properties of the IAA-over-producing mutant strains to the parent strain.

Whole broth from mutant strains and from the parent strain were prepared for use as a drench or seed treatment. The seed flask containing Luria Broth (LB) was inoculated and grown for 7-9 hours at 35° C. The next day, 5 mL of the seed flask was inoculated into a high protein medium. The flask grew at 35° C. until sporulation was complete.

### Cell Tray Assays

Plug trays (200 sq. in.) were filled with seed germination potting mix, and each cell was seeded with one corn seed. Each cell was then treated with 2 mL of a 20% dilution of the above-described whole broth samples, such that each cell received at least 1×10⁸ CFU of the parent or mutant strain. The untreated control cells received 2 mL of water. These trays were placed on growth racks under high-intensity lights set to a 14-hour light/10-hour dark schedule) at room temperature. Watering was done as needed. No fertilizer was used.

Corn plants were observed for plant growth promotion traits two weeks after drenching the seeds. The leaf and root tissues were harvested and weighed. The harvested fresh plant weight from plants treated with the mutant strain as compared to plants treated with the parent strain was 116%, where fresh weight from plants treated with the parent strain was set at 100%.

### Greenhouse Assays

Greenhouse assays were conducted in a similar manner to the cell tray assays, using seed germination potting mix in large pots rather than cell trays. Whole broth was prepared as described above, optical density measured and seeds coated with about 1×10⁶ to about 1×10⁷ CFU/seed and dried.

Corn plants were observed for plant growth promotion four weeks after treating the seeds. The leaf and root tissues were harvested, dried in an oven at 80° C., and weighed one week after drying. The dry plant weight from plants treated with the mutant strain as compared to plants treated with the parent strain was 109%, where dry plant weight from plants treated with the parent strain was set at 100%.

### Field Trials

Field trials were conducted in soybean to compare plant growth promotion capabilities of the mutant strain and the parent strain. Specifically, the parent and mutant strains were cultured under the same conditions in a high protein media until sporulation. The resulting whole broths were freeze dried and applied to the soybean seed. Specifically, 1×10⁷ CFU of each strain were applied per soybean seed. . . . Such treated soybean seeds were planted and grown during the normal season until harvest. NRRL B-67003-treated seeds showed a 9.9% increase in yield compared to the untreated control (which was set at 100%). Seeds treated with the parent strain showed a 4.9% increase in yield compared to the untreated control.

Additional field trials were conducted in corn to determine plant growth promotion capabilities of the mutant strain. Freeze-dried whole broths of the parent and mutant strain were prepared as described above and applied to seeds along with a base chemical package as described in Table 1 below.

TABLE 1

| Seed | | |
|---|---|---|
| Treatment | Compound | Application Rate |
| Base | ALLEGIANCE FL | 2 gal. active ingredient/ |
| | (Active ingredient: | 100 kg seed |
| | metalaxyl - 28.35%) | |
| | PONCHO 600 | 0.11 mg active ingredient/ |
| | (Active ingredient = | seed |
| | clothianidin) | |
| | PRO-IZED RED | 32.6 mL/100 kg seed |
| | Seed Colorant | |
| | PERIDIAM PRECISE | 65 mL/100 kg seed |
| | Seed Finisher 1010 | |
| Biological | NRRL B-67003 or | 0.31 mL/1000 seeds |
| | I-1582 | (1 × 10⁷ CFU/seed) |

Nine trials were conducted in in corn, each containing two plots treated with NRRL B-67003 and one plot treated with the parent strain. Average percent yield gains over the treated control, which received the base treatment only, are shown in Table 2, below.

TABLE 2

| | Average Percent Yield Over |
|---|---|
| Treatment | Base-Treated Control |
| Base + NRRL B-67003 | 3.0% |
| Base + I-1582 | 2.0% |

### Example 4

### Cell Fusion to Generate Improved Mutants

Mutants of Bacillus firmus I-1582, which showed enhanced production of IAA and improved plant growth promotion properties, were selected for genome shuffling and subjected to the following protocol. Individual B. firmus mutants were cultured for 16-24 hours at 37° C., 220 rpm in 14 mL culture tubes containing 5 mL of Luria Broth (LB), pH 8. When the culture's O.D. at 600 nm of 0.6 was reached, cells were centrifuged at 4000×g for 10 minutes at room temperature. SMMP buffer was prepared by mixing four parts Penassay buffer (1.5 g/L Bacto-Beef extract; 1.5 g/L Bacto-Yeast Extract; 5 g/L Bacto-Peptone; 1 g/L Bacto-Dextrose; 3.5 g/L NaCl; 3.68 g/L K₂HPO₄:1.32 g/L KH₂PO₄, pH 7) and two parts SMM buffer (0.5 M Sucrose; 20 mM Maleate; 20 mM MgCl₂, pH 6.5). Cell pellet was resuspended in SMMP containing 0.2 mg/mL egg white lysozyme and incubated for 1 hour at 37° C. A phase-contrast microscope was used to monitor protoplasting efficiency. Protoplasted cells were centrifuged at 4000×g for 10 minutes at 4° C. and washed twice with SMMP buffer and finally resuspended in SMMP buffer equal to 1/10 the original culture volume. Individual protoplasted mutant strains were then pooled together in equal volumes. 900 μL of PEG solution (SMM buffer with 40% PEG 6000 v/v) was then added to protoplast pool and incubated for 2 minutes at room temperature. Following incubation, fusants were plated on SA5 agar (0.5 M Na Succinate; 0.5% Casamino Acids w/v; 0.5% Yeast Extract w/v; 30 mM MgCl₂; 12.5 mM CaCl₂; % NaCl w/v; 0.5% Glucose; 10 mM TRIS, pH 8, 5% Agar w/v). Genome shuffling fusants were then screened or subjected to recursive shuffling prior to screening. For recursive shuffling, fused protoplasts were plated on SA5 agar and incubated at 37° C. for 16 hours. Cells were scraped from agar plate, and subjected to another round of protoplast fusion and recovery. This process was repeated one or more additional times. Individual clones were selected and screened for IAA production, protease activity and superoxide dismutase (“SOD”) activity.

IAA production by NRRL B-67518 was measured as described in Example 2, above. Bacillus firmus NRRL B-67518 showed a 3.7 fold increase in IAA production compared to the parent strain (i.e., I-1582).

To assess protease activity, cells were pelleted from whole broth culture. 50 μL of supernatant was collected and added to 400 μL of 0.24% AzoCasein solution and incubated for 2 hrs at 35° C. 12% TCA was then added and reaction was subsequently centrifuged at 6000×g for 5 minutes. Supernatant fraction was collected and added to 2 N NaOH. Absorbance was then measured at 450 nm and protease activity assessed. Protease activity by NRRL B-67518 showed a 1.2 fold increase compared to the parent strain (i.e., I-1582).

To measure SOD activity, cultures were assayed using an SOD assay kit from Sigma Aldrich (#19160). SOD activity by NRRL B-67518 showed a 1.3 fold increase compared to the parent strain (i.e., I-1582).

### Example 5

### Additional Cell Tray Assays, including NRRL B-67518

Cell tray assays were conducted as described in Example 3, above. Each cell that contained a seed was drenched with 2 mL of 20% whole broth solution to deliver 1×10⁷ CFU/mL. Untreated controls were drenched with water. Bacillus firmus NRRL B-67518 increased plant biomass by 15.7% over the untreated control, while the parent strain, I-1582, increased plant biomass by 10.68% over the untreated control. In addition, it was observed that the corn plants treated with NRRL B-67518 had broader leaves and denser foliage than the untreated control and the plants treated with the parent strain.

Long-term yield studies were conducted in corn in the greenhouse using NRRL B-67003, NRRL B-67518 and the parent strain, I-1582. Whole broth of each strain was prepared as described above and seeds treated to deliver 1×10⁷ CFU/seed. The seeds were planted in a 3.5 gallon pot filled with potting media. Twelve replications were planted to count for germination differences and data was collected from eight or nine plants at the end of the study. Plants received daily watering through a drip line. Fertilizer was also added with water one time a week until flowering. The total study duration was 93 days after which mature cobs were collected, dried, and seeds were removed. Per plant seed yield was recorded in terms of grams per plant. The results were expressed in terms of % increase over seeds treated with the parent strain. The parent strain also increased growth over the untreated control. Table 3, below, shows percent increases in grain weight for plants treated with the mutant strains over the plants treated with the parent strain.

TABLE 3

| | Percent Increase Over |
|---|---|
| Treatment | Parent-Treated Seeds |
| NRRL B-67003 | 14.1 |
| NRRL B-67518 | 21.9 |

Unless defined otherwise, all technical and scientific terms herein have the same meaning as commonly understood by one of ordinary skill in the art to which this invention belongs. All publications, patents, and patent publications cited are incorporated by reference herein in their entirety for all purposes.

It is understood that the disclosed invention is not limited to the particular methodology, protocols and materials described as these can vary. It is also understood that the terminology used herein is for the purposes of describing particular embodiments only and is not intended to limit the scope of the present invention which will be limited only by the appended claims.

We claim:

1. A composition comprising a biologically pure culture of a mutant of Bacillus firmus I-1582 having improved indole acetic acid production and improved ability to promote plant growth compared to Bacillus firmus I-1582.

2. The composition of claim 1, wherein the mutant comprises a biologically pure culture of a Bacillus firmus strain NRRL B-67003, a Bacillus firmus strain NRRL B-67518 or a mutant having all the identifying characteristics of one or more of the strains and improved indole acetic acid production compared to one or more of the strains.

3. The composition of claim 2, wherein the mutant has improved ability to promote plant growth compared to Bacillus firmus strain NRRL B-67003 or Bacillus firmus strain NRRL B-67518.

4. The composition according to claim 1, wherein the mutant strain has a genomic sequence with greater than about 90% sequence identity to Bacillus firmus strain I-1582.

5. The composition of claim 4, wherein the mutant strain has the ability to increase plant yield or total plant weight that is better than that of Bacillus firmus I-1582.

6. The composition according to claim 2, wherein the mutant strain has a genomic sequence with greater than about 90% sequence identity to Bacillus firmus I-1582, Bacillus firmus strain NRRL B-67003 or Bacillus firmus strain NRRL B-67518.

7. The composition of claim 6, wherein the mutant has the ability to increase plant yield or total plant weight that is better than that of Bacillus firmus I-1582, Bacillus firmus NRRL B-67003 or Bacillus firmus NRRL B-67518.

8. The composition of claim 1 or claim 2, wherein the mutant strain has nematode control activity that is comparable to or better than that of Bacillus firmus I-1582.

9. The composition of any one of the preceding claims further comprising an agriculturally acceptable carrier.

10. A composition comprising a fermentation product of Bacillus firmus strain NRRL B-67003, Bacillus firmus strain NRRL B-67518, or a mutant of one or more of the strains having all the identifying characteristics of one or more of the strains.

11. The composition of claim 10, wherein the fermentation product further comprises a formulation ingredient.

12. The composition of claim 11, wherein the formulation ingredient is a wetting agent.

13. The composition of claim 10, wherein the fermentation product is a freeze-dried powder or a spray-dried powder.

14. The composition of claim 13 comprising at least about 1×10⁶ CFU of the strain/g freeze-dried powder or spray-dried powder.

15. The composition of claim 10, wherein the fermentation product is a liquid formulation.

16. The composition of claim 15, wherein the liquid formulation is a suspension concentrate.

17. The composition of claim 15 or 16 comprising at least about 2×10⁹ CFU of the strain/mL of the liquid formulation.

18. A method of treating a plant to enhance plant growth, wherein the method comprises applying a Bacillus firmus strain I-1582 or a mutant derived from the strain having all the identifying characteristics of the strain and improved ability to produce indole acetic acid and to promote plant growth, to the plant, to a part of the plant and/or to a locus of the plant.

19. The method of claim 18, wherein the mutant is Bacillus firmus strain NRRL B-67003, Bacillus firmus strain NRRL B-67518, or a strain derived from one or more of the strains having all the identifying characteristics of one or more of the strains.

20. The method of claim 19, wherein the composition is a fermentation product of the Bacillus firmus strain NRRL B-67003, the Bacillus firmus strain NRRL B-67518 or the mutant.

21. The method of claim 18 or claim 19, wherein the method comprises applying the composition to seed.

22. The method of claim 18 or claim 19, wherein the composition is applied at about 1×10⁵ to about 1×10⁸ colony forming units (CFU) per seed of any of the strains.

23. The method of claim 18 or claim 19, wherein the composition is applied to soil at about 1×10¹³ to about 1×10¹⁵ colony forming units (CFU) per hectare of any of the strains.

24. The method according to any one of claims 18 to 23, wherein yield of the plant is increased by at least 1%.

25. The method of any one of the above claims, wherein the plant is selected from the group consisting of cotton, corn, sorghum, soybeans and sugarbeet.

26. A method for generating a mutant of one or more parent Bacillus firmus strains with improved plant growth promotion properties comprising mutagenizing the one or more parent Bacillus firmus strains to create mutants, measuring production of indole acetic acid in the mutants, selecting a mutant with enhanced production of indole acetic acid compared to the one or more parent Bacillus firmus strains, and producing a fermentation product of the selected mutant.

27. The method of claim 26, wherein the parent Bacillus firmus strain is strain I- 1582.

28. The method of claim 27, wherein the mutagenizing comprises chemical mutagenesis of Bacillus firmus strain I-1582.

29. The method of claim 26 further comprising a second mutagenizing step after selection of a mutant with enhanced production of indole acetic acid compared to the one or more parent Bacillus firmus strains.

30. The method of claim 26, wherein the step of mutagenizing is repeated one or more times with the one or more mutants created by the previous mutagenizing step.

31. The method of claim 30, wherein the mutagenizing comprises genome shuffling with two or more mutants created by the previous mutagenizing step.

32. The method of claim 26 further comprising applying the fermentation product of the one or more mutants to a plant, applying fermentation product of the one or more parent Bacillus firmus strains to a plant, measuring one or more plant growth promotion properties and selecting the mutant that has one or more improved plant growth promotion properties compared to the one or more parent strains.

33. The method of claim 32, wherein the measured plant growth promotion property is ability to increase plant weight.

34. The method of claim 32, wherein the measured plant growth promotion property is ability to increase plant yield.

35. The method of claim 33, wherein plant weight is increased by at least 1%.

36. The method of claim 34, wherein plant yield is increased by at least 1%.

37. The method of claim 32 further comprising mutagenizing the mutants having one or more improved plant growth promotion properties.

38. The method of claim 37, wherein the step of mutagenizing the mutants having one or more improved plant growth promotion properties is repeated one or more times with the one or more mutants created by the previous mutagenizing step.

39. The method of claim 38, wherein the mutagenizing comprises genome shuffling with two or more mutants created by the previous mutagenizing step.

40. The method of claim 32, wherein the measuring further comprises measuring enzyme activity of protease and/or superoxide dismutase and wherein the selecting further comprises selecting a mutant with enhanced enzyme activity of protease and/or superoxide dismutase.
